(19) United States
(12) Patent Application Publication
(10) Pub. No.: US 2021/0074411 A1
(43) Pub. Date: Mar. 11, 2021

(54) SYSTEM, METHOD AND COMPUTER-ACCESSIBLE MEDIUM FOR A PATIENT SELECTION FOR A DUCTAL CARCINOMA IN SITU OBSERVATION AND DETERMINATIONS OF ACTIONS BASED ON THE SAME

(71) Applicant: THE TRUSTEES OF COLUMBIA UNIVERSITY IN THE CITY OF NEW YORK, New York, NY (US)

(72) Inventor: Richard HA, White Plains, NY (US)

(21) Appl. No.: 16/950,043

(22) Filed: Nov. 17, 2020

Related U.S. Application Data
(63) Continuation of application No. PCT/US2019/032946, filed on May 17, 2019.
(60) Provisional application No. 62/672,945, filed on May 17, 2018.

(51) Int. Cl. G16H 30/40; G16H 50/20; G06T 7/00

(52) U.S. Cl. CPC G16H 30/40; G16H 50/20; G06T 2207/10088; G06T 2207/30068; G06T 7/0014

(57) ABSTRACT

An exemplary system, method and computer-accessible medium for determining ductal carcinoma in situ (DCIS) information regarding a patient(s) can include for example, receiving image(s) of internal portion(s) of a breast of the patient(s), and automatically determining the DCIS information by applying a neural network(s) to the image(s). The DCIS information can include predicting (i) pure DCIS or (ii) DCIS with invasion. Input information of the patient(s) can be selected for a DCIS observation for determining the DCIS information. The image(s) can be a mammographic image(s). The image(s) can be one of a magnetic resonance image or a computer tomography image.

### CROSS-REFERENCE TO RELATED APPLICATION(S)

This application relates to and claims priority from U.S. Patent Application No. 62/672,945, filed on May 17, 2018, the entire disclosure of which is incorporated herein by reference.

### FIELD OF THE DISCLOSURE

The present disclosure relates generally to Ductal Carcinoma observation and/or determination, and more specifically, to exemplary embodiments of exemplary system, method and computer-accessible medium for patient selection for Ductal Carcinoma in Situ observation and/or determination of possible actions based on the same.

### BACKGROUND INFORMATION

Attempts to minimize over-diagnoses and treatment of Ductal Carcinoma in Situ (“DCIS”) have led to clinical trials of observing patients with DCIS instead of surgery. Despite careful selection for “low risk” DCIS patients, occult invasive cancers can occur in significant number of these patients.

Thus, it may be beneficial to provide an exemplary system, method and computer-accessible medium for patient selection for ductal carcinoma in situ observation and/or determination of possible actions based on the same which can overcome at least some of the deficiencies described herein above.

### SUMMARY OF EXEMPLARY EMBODIMENTS

An exemplary system, method and computer-accessible medium for determining ductal carcinoma in situ (DCIS) information regarding a patient(s) can include for example, receiving image(s) of internal portion(s) of a breast of the patient(s), and automatically determining the DCIS information by applying a neural network(s) to the image(s). The DCIS information can include predicting (i) pure DCIS or (ii) DCIS with invasion. Input information of the patient(s) can be selected for a DCIS observation for determining the DCIS information. The image(s) can be a mammographic image(s). The image(s) can be one of a magnetic resonance image or a computer tomography image.

In some exemplary embodiments of the present disclosure, the image(s) can contain a calcification(s). The image can be segmented and/or resized. The image can be centered using a histogram-based z score normalization of non-air pixel intensity values. The image(s) can be (i) randomly flipped, (ii) randomly rotated, or (iii) randomly cropped. A random affine shear can be applied to the image(s). The neural network(s) can be a convolutional neural network (CNN). The CNN can include a plurality of layers. The CNN can include 15 hidden layers. The CNN can include five residual layers. The CNN can include an inception style layer(s) after a ninth hidden layer. The CNN can include a fully connected layer(s) after a 13^{th} layer thereof. The fully connected layer(s) can include 16 neurons. The CNN can include a linear layer(s) after a 13^{th} layer. The linear layer(s) can include 8 neurons. A determination can be made as to what action to perform or whether to perform any action based on the determined DCIS information.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIGS. 1A-1C are exemplary input images for the exemplary convolutional neural network of patients with DCIS according to an exemplary embodiment of the present disclosure;

FIG. 2 is an exemplary diagram of the exemplary convolutional neural network according to an exemplary embodiment of the present disclosure;

FIG. 3 is an exemplary flow diagram of an exemplary method for determining DCIS information regarding a patient according to an exemplary embodiment of the present disclosure; and

FIG. 4 is an illustration of an exemplary block diagram of an exemplary system in accordance with certain exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments and is not limited by the particular embodiments illustrated in the figures and the appended claims.

### DETAILED DESCRIPTION

### Exemplary Definitions

Conventional neural networks: Conventional neural networks can be, but not limited to, networks composed of neurons with learnable weights and biases. Raw data (e.g., an image) is input into the machine, which encodes defining characteristics into the network architecture. Each neuron receives multiple inputs, calculates a weighted sum that goes through an activation function, and creates an output.

Convolutional layer: The convolutional layer can apply a filter that slides over the entire image to calculate the dot product of each particular region. In this procedure, one image can become a stack of filtered images.

Pooling layer: The pooling layer can reduces the spatial size of each feature map. Maximum pooling can apply a filter that slides over the entire image and keeps only the maximum value for each particular region.

Rectified linear units: Rectified linear units can be, but not limited to, computation units that perform normalization of the stack of images. In a rectified linear unit, for example, all negative values can be changed to zero.

Inception layer: The inception layer can reduce the computation burden by making use of dual computational layers.

Fully connected layer: In the fully connected layer, as an example, every feature value from the created stack of filtered images can have a weighted output, which can be averaged to create a prediction.

Back propagation: In back propagation, the error of the final prediction can be calculated, and can be used to adjust each feature value to improve future predictions.

Dropout: Dropout can be, but not limited to, a regularization procedure used to reduce overfitting of the network by preventing coadaptation of training data. Dropout randomly selects neurons to be ignored during training.

L2 regularization: L2 regularization can be, but not limited to, a regularization procedure used to reduce overfitting by decreasing the weighted value of features to simplify the model.

The exemplary system, method, and computer-accessible medium, according to an exemplary embodiment of the present disclosure, can utilize a convolutional neural network (“CNN”) for predicting patients with pure DCIS versus DCIS with invasion using, for example, mammographic images; however, it should be understood that other imaging modalities can be used.

### Exemplary Procedures and Methods

A retrospective study utilizing the exemplary CNN was performed, which included 246 unique images from 123 patients. Additionally, 164 images in 82 patients diagnosed with DCIS by stereotactic-guided biopsy of calcifications without any upgrade at the time of surgical excision (e.g., pure DCIS group) were used. 82 images in 41 patients with mammographic calcifications yielding occult invasive carcinoma as the final upgraded diagnosis on surgery (e.g., occult invasive group) were used. Two mammographic magnification views (e.g., bilateral craniocaudal and mediolateral/lateralmedial) of the calcifications were used for analysis. Calcifications were segmented using an exemplary 3D Slicer, which were then resized to fit a 128×128 pixel bounding box. A 15 hidden layer topology was used to implement the exemplary CNN. The exemplary network architecture included 5 residual layers and a dropout of 0.25 after each convolution. Cases were randomly separated into a training set (e.g., 80%) and a validation set (e.g., 20%).

### Exemplary Data Preparation

An original pathology report was determined to be ground truth information and was used as the basis for dividing patients. Eighty percent of the available patients were randomly selected to develop the exemplary network, and the remaining 20% of patients were used to test the exemplary CNN.

### Exemplary Data Augmentation and Segregation

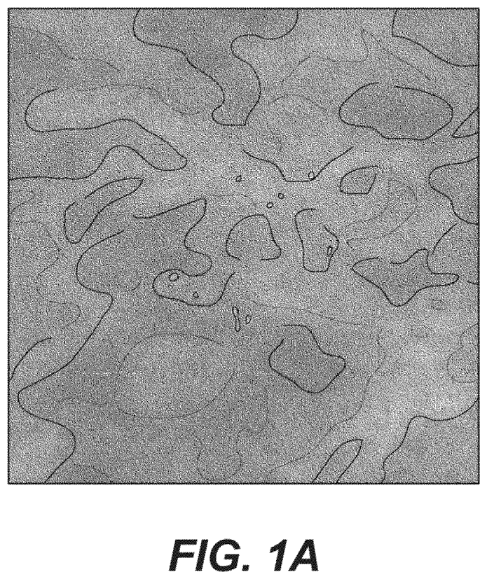

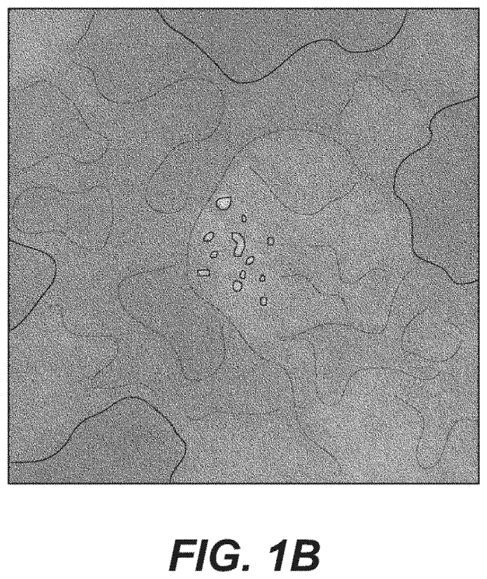

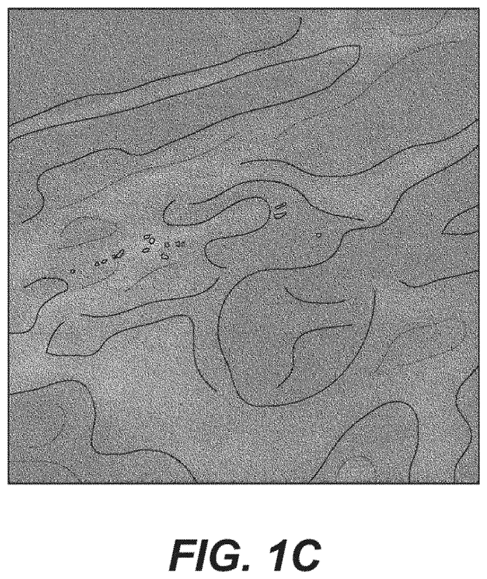

The magnification views of each patient's mammogram were loaded into a 3D segmentation program. Segments were extracted using an exemplary automatic segmentation procedure to include the regions of the magnification view that contained calcifications. Each image was scaled in size on the basis of the radius of the segmentations and was resized to fit a bounding box of 128×128 pixels. FIGS. 1A-1C illustrate exemplary input images for the exemplary CNN of patients with DCIS according to an exemplary embodiment of the present disclosure. The entire image batch was centered using histogram-based z score normalization of the non-air pixel intensity values. Exemplary data augmentation was performed to limit overfitting. Some of the magnification views (e.g., orthogonal magnification views) were randomly flipped vertically, horizontally, or in both directions. Additionally, some of the magnification views were rotated by a random angle between 0.52 and −0.52 radians, and were randomly cropped to a box 80% of the initial size. Random affine shear was applied to each input image.

### Exemplary Network Architecture

A topology with multiple layers, for example, 15 hidden layers, can be used to implement the exemplary CNN. The exemplary CNN can include fully convolutional (“FC”) layers. The exemplary CNN can include the application of a series of convolutional matrices to a vectorized input image that can iteratively separate the input to a target vector space. The exemplary CNN can include five residual layers. The residual neural networks can be used to stabilize gradients during back propagation, facilitating improved optimization and greater network depth. For example, starting with the 10th hidden layer, inception V2 style layers can be used. The inception layer architecture can facilitate a computationally efficient procedure for facilitating a network to selectively determine the appropriate filter architectures for an input feature map, providing improved learning rates.

A fully connected layer with, for example, 16 neurons can be implemented after, as an example, the 13th hidden layer, which can be followed by implantation of a linear layer with eight neurons. A final softmax function output layer with two classes can be inserted as the last layer. Training was performed using an exemplary optimization procedure (e.g., the AdamOptimizer optimization procedure) (see, e.g., Reference 20), combined with an exemplary accelerated gradient procedure (e.g., the Nesterov accelerated gradient procedure). (See, e.g., References 21 and 22). Parameters were initialized using an exemplary heuristic. (See, e.g., Reference 23). L2 regularization was performed to prevent over-fitting of data by limiting the squared magnitude of the kernel weights. Dropout (e.g., 25% randomly) was also used to prevent overfitting by limiting unit coadaptation. (See, e.g., Reference 24). Batch normalization was used to improve network training speed and regularize performance by reducing internal covariate shift. (See, e.g., Reference 25).

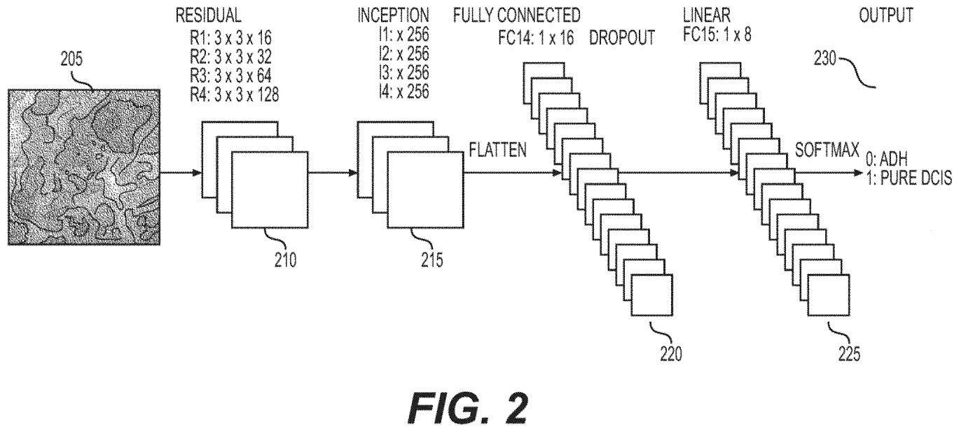

FIG. 2 shows an exemplary diagram of the exemplary CNN according to an exemplary embodiment of the present disclosure. For example, as shown in FIG. 2, a DCIS image 205 can be input into the exemplary CNN. Image 205 can be input into a set of residual layers 210 (e.g., four layers, which can include R1: 3×3×16; R2: 3×3×32; R3: 3×3×64; and R4: 3×3×128). A plurality of inception layers 215 can be used (e.g., four inception layers, which can include I1: ×256; I2: ×256; I3: ×256: and I4: ×256). Multiple fully connected layers 220 can be implemented (e.g., 15 fully connected layers, which can include one or more fully connected layers, for example, FC14: 1×16 dropout). Additionally, multiple linear layers 225 can be used (e.g., 15 linear layers, which can include one or more fully connected layers, for example, FC: 1×8). The Exemplary CNN can produce an output 230, which can be used, for example, to (i) predict pure DCIS or DCIS with invasion and/or (ii) select a patient for DISC.

Softmax with cross-entropy hinge loss was used as the primary objective function of the network to provide a more intuitive output of normalized class probabilities. A class-sensitive cost function penalizing incorrect classification of the underrepresented class was used. A final softmax score threshold of 0.5 from the mean of raw logits from the ML and CC views was used for two-class classification. The area under the curve (“AUC”) value was used as the primary performance metric. Sensitivity, specificity, and accuracy were also calculated as secondary performance metrics.

Visualization of network predictions was performed using an exemplary gradient-weighted class activation mapping (“Grad-CAM”) procedure. (See, e.g., Reference 26). Each Grad-CAM map was generated by an exemplary prediction model along with every input image. The salient region of the averaged Grad-CAM map illustrates where important features come from when the exemplary prediction model makes classification decisions.

### Exemplary Results

The exemplary CNN procedure for predicting patients with pure DCIS achieved an overall accuracy of about 74.6% (e.g., about 95% CI, ±5) with area under the ROC curve of about 0.71 (e.g., about 95% CI, ±0.04), a specificity of about 49.4% (e.g., about 95% CI, ±6%) and a sensitivity of about 91.6% (e.g., about 95% CI, ±5%).

Thus, as described above, the exemplary system, method, and computer-accessible medium, according to an exemplary embodiment of the present disclosure, can utilize the exemplary CNN to distinguish pure DCIS from DCIS with invasion using, for example, using mammographic images.

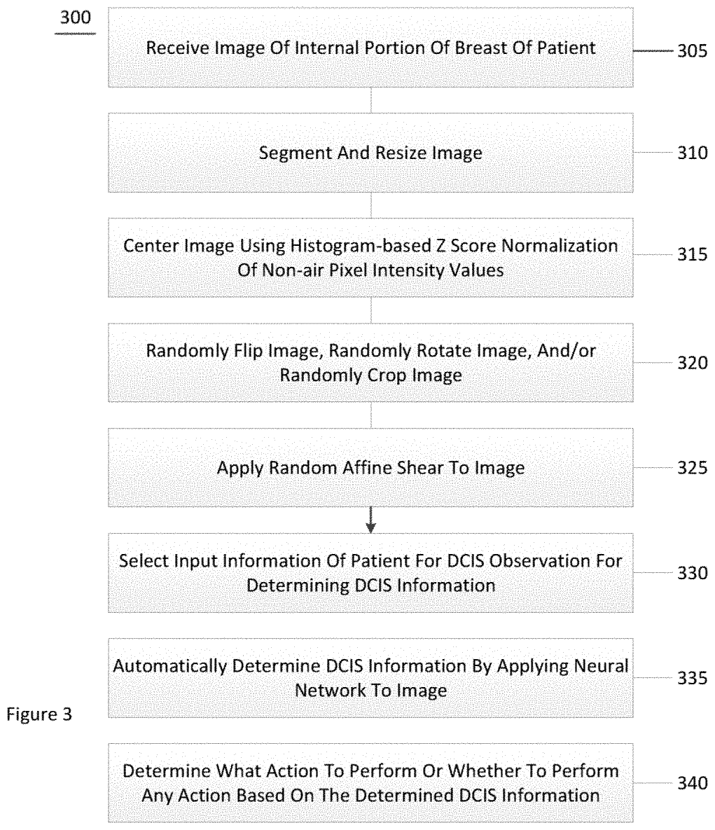

FIG. 3 shows an exemplary flow diagram of an exemplary method 300 for determining DCIS information regarding a patient according to an exemplary embodiment of the present disclosure. For example, at procedure 305, an image of an internal portion of a breast of a patient can be received. At procedure 310, the image can be segmented and resized. At procedure 315, the image can be centered using a histogram-based z score normalization of non-air pixel intensity values. At procedure 320, the image can be randomly flipped, randomly rotated, and/or randomly cropped. At procedure 325, a random affine shear can be applied to the image. At procedure 330, input information of patient for DCIS observation can be selected for determining DCIS information. At procedure 335, DCIS information can be automatically determined by applying a neural network to the image. At procedure 340, a determination can be made as to what action to perform or whether to perform any action based on the determined DCIS information.

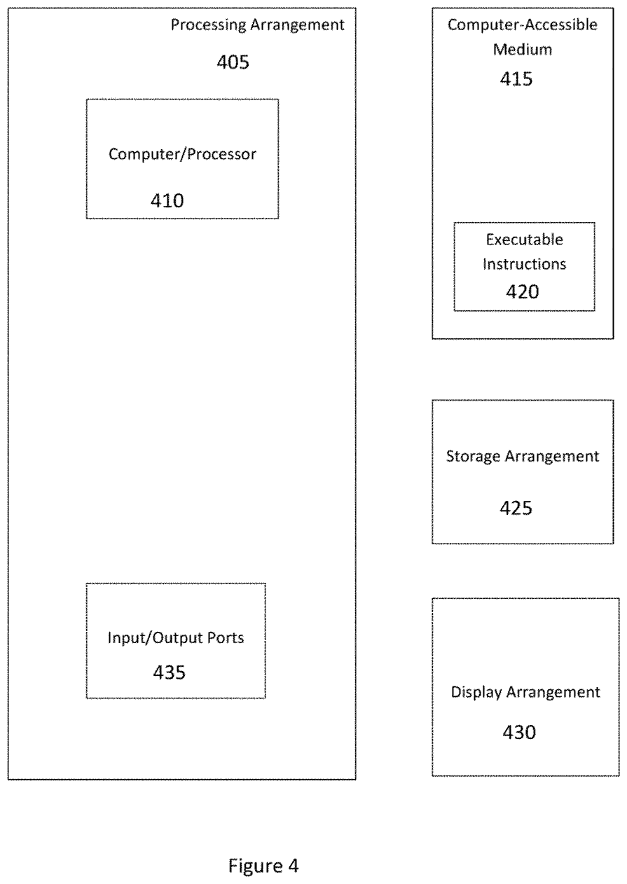

FIG. 4 shows a block diagram of an exemplary embodiment of a system according to the present disclosure. For example, exemplary procedures in accordance with the present disclosure described herein can be performed by a processing arrangement and/or a computing arrangement (e.g., computer hardware arrangement) 405. Such processing/computing arrangement 405 can be, for example entirely or a part of, or include, but not limited to, a computer/processor 410 that can include, for example one or more microprocessors, and use instructions stored on a computer-accessible medium (e.g., RAM, ROM, hard drive, or other storage device).

As shown in FIG. 4, for example a computer-accessible medium 415 (e.g., as described herein above, a storage device such as a hard disk, floppy disk, memory stick, CD-ROM, RAM, ROM, etc., or a collection thereof) can be provided (e.g., in communication with the processing arrangement 405). The computer-accessible medium 415 can contain executable instructions 420 thereon. In addition or alternatively, a storage arrangement 425 can be provided separately from the computer-accessible medium 415, which can provide the instructions to the processing arrangement 405 so as to configure the processing arrangement to execute certain exemplary procedures, processes, and methods, as described herein above, for example.

Further, the exemplary processing arrangement 405 can be provided with or include an input/output ports 435, which can include, for example a wired network, a wireless network, the internet, an intranet, a data collection probe, a sensor, etc. As shown in FIG. 4, the exemplary processing arrangement 405 can be in communication with an exemplary display arrangement 430, which, according to certain exemplary embodiments of the present disclosure, can be a touch-screen configured for inputting information to the processing arrangement in addition to outputting information from the processing arrangement, for example. Further, the exemplary display arrangement 430 and/or a storage arrangement 425 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

The following references are hereby incorporated by reference in their entireties, as follows:
[1] Nguyen C V, Albarracin C T, Whitman G J, Lopez A. Sneige N. Atypical ductal hyperplasia in directional vacuum-assisted biopsy of breast microcalcifications: considerations for surgical excision. Ann Surg Oncol 2011: 18:752-761.
[2] Sinn H P, Kreipe H. A brief overview of the WHO classification of breast tumors, 4th edition, focusing on issues and updates from the 3rd edition. Breast Care (Basel) 2013.
[3] Racz J M, Carter J M, Degnim A C. Lobular neoplasia and atypical ductal hyperplasia on core biopsy: current surgical management recommendations. Ann Surg Oncol 2017; 24:2848-2854.
[4] Ko E, Han W, Lee J W, et al. Scoring system for predicting malignancy in patients diagnosed with atypical ductal hyperplasia at ultrasound-guided core needle biopsy. Breast Cancer Res Treat 2008; 112:189-195.
[5] Menen R S, Ganesan N, Bevers T, et al. Long-term safety of observation in selected women following core biopsy diagnosis of atypical ductal hyperplasia. Ann Surg Oncol 2017; 24:70-76.
[6] Pankratz V S, Hartmann L C, Degnim A C, et al. Assessment of the accuracy of the Gail model in women with atypical hyperplasia. J Clin Oncol 2008; 26:5374-5379.
[7] Deshaies I, Provencher L, Jacob S, et al. Factors associated with upgrading to malignancy at surgery of atypical ductal hyperplasia diagnosed on core biopsy. Breast 2011; 20:50-55.
[8] Bendifallah S, Defert S, Chabbert-Buffet N, et al. Scoring to predict the possibility of upgrades to malignancy in atypical ductal hyperplasia diagnosed by an 11-gauge vacuum-assisted biopsy device: an external validation study. Eur J Cancer 2012; 48:30-36.
[9] Yu Y H, Liang C, Yuan X Z. Diagnostic value of vacuum-assisted breast biopsy for breast carcinoma: a meta-analysis and systematic review. Breast Cancer Res Treat 2010; 120:469-479.
[10] Song J L, Chen C, Yuan J P, Sun S R. Progress in the clinical detection of heterogeneity in breast cancer. Cancer Med 2016; 5:3475-3488.
[11] Gomes D S, Porto S S, Balabram D, Gobbi H. Inter-observer variability between general pathologists and a specialist in breast pathology in the diagnosis of lobular neoplasia, columnar cell lesions, atypical ductal hyperplasia and ductal carcinoma in situ of the breast. Diagn Pathol 2014; 9:121.
[12] Ha R, Chang P, Mutasa S, et al. Convolutional neural network using a breast MRI tumor dataset can predict Oncotype Dx recurrence score. J Magn Reson Imaging 2018 Aug. 21.
[13] Ha R, Chang P, Mema E, et al. Fully automated convolutional neural network method for quantification of breast MRI fibroglandular tissue and background parenchymal enhancement. J Digit Imaging 2018 Aug. 3.
[14] Ha R, Chang P, Karcich J, et al. Convolutional neural network based cancer risk stratification using a mammographic dataset. Acad Radiol 2018 Jul. 31.
[15] Ribli D, Horváth A, Unger Z, Pollner P, Csabai I. Detecting and classifying lesions in mammograms with Deep Learning. Sci Rep 2018; 8:4165.
[16] Mohamed A A, Berg W A, Peng H, Luo Y, Jankowitz R C, Wu S. A deep learning method for classifying mammographic breast density categories. Med Phys 2018; 45:314-321.
[17] LeCun Y, Bottou L, Bengio Y, Haffner P. Gradient-based learning applied to document recognition. Proc IEEE 1998; 11:2278-2324.
[18] He K, Zhang X, Ren S, Sun J. Deep residual learning for image recognition. Xplore Digital Library website. ieeexplore.ieee.org/document/7780459. Published 2016.
[19] Szegedy C, Liu W, Jia Y, et al. Going deeper with convolutions. Xplore Digital Library website. ieeexplore.ieee.org.document/7298594IEEE. Published 2015.
[20] Kingma D P. Ba J, Adam: a method for stochastic optimization. arXiv website. arxiv.org/abs/1412.6980. Published 2014.
[21] Nesterov Y, Gradient methods for minimizing composite objective function. Optimization Online website. www.optimization-online.org/DB_FILE/2007/09/1784.pdf. Published 2007.
[22] Dozat T, Incorporating Nesterov momentum into Adam. Stanford University website. cs229.stanford.edu/proj2015/054_report.pdf. Published 2016.
[23] Glorot X, Bengio Y, Understanding the difficulty of training deep feedforward neural networks. Proceedings of Machine Learning Research website. proceedings.mlr.press/v9/glorot10a/glorot10a.pdf. Published 2010.
[24] Srivastava N, Hinton G E, Krizhevsky A, et al. Dropout: a simple way to prevent neural networks from overfitting. J Mach Learn Res 2014; 15:1929-1958.
[25] Ioffe S, Szegedy C. Batch normalization: accelerating deep network training by reducing internal covariate shift. Proceedings of Machine Learning Research website. proceedings.mlr.press/v37/ioffe15.html. Published 2015.
[26] Selvaraju R R, Das A, Vedantam R, et al. Grad-CAM: why did you say that? Visual explanations from deep networks via gradient-based localization. arXiv website. arxiv.org/abs/1610.02391v1. Published 2016.
[27] Araújo T, Aresta G, Castro E, et al. Classification of breast cancer histology images using convolutional neural networks. PLoS One 2017; 12:e0177544.
[28] Bejnordi B E, Zuidhof G, Balkenhol M, et al. Context-aware stacked convolutional neural networks for classification of breast carcinomas in whole-slide histopathology images. J Med Imaging (Bellingham) 2017; 4:044504.
[29] Tsuchiya K, Mori N, Schacht D V, et al. Value of breast MRI for patients with a biopsy showing atypical ductal hyperplasia (ADH). J Magn Reson Imaging 2017; 46:1738-1747.
[30] Menes T, Kerlikowske K, Jaffer S, et al. Rates of atypical ductal hyperplasia have declined with less use of postmenopausal hormone treatment: findings from the Breast Cancer Surveillance Consortium. Cancer Epidemiol Biomarkers Prev 2009; 18:2822-2828.

1. A non-transitory computer-accessible medium having stored thereon computer-executable instructions for determining ductal carcinoma in situ (DCIS) information regarding at least one patient, wherein, when a computer arrangement executes the instructions, the computer arrangement is configured to perform procedures comprising:
receiving at least one image of at least one internal portion of a breast of the at least one patient; and
automatically determining the DCIS information by applying at least one neural network to the at least one image.

2-20. (canceled)

21. A method for determining ductal carcinoma in situ (DCIS) information regarding at least one patient, comprising:
receiving at least one image of at least one internal portion of a breast of the at least one patient; and
using a computer hardware arrangement, automatically determining the DCIS information by applying at least one neural network to the at least one image.

22. The method of claim 21, wherein the DCIS information includes predicting (i) pure DCIS or (ii) DCIS with invasion.

23. The method of claim 21, further comprising selecting input information of the at least one patient for a DCIS observation for determining the DCIS information.

24. The method of claim 21, wherein the at least one image is at least one of (i) at least one mammographic image, (ii) a magnetic resonance image, or (iii) a computer tomography image.

25. (canceled)

26. The method of claim 21, wherein the at least one image contains at least one calcification.

27. The method of claim 21, further comprising segmenting and resizing the at least one image.

28. The method of claim 27, further comprising centering the at least one image using a histogram-based z score normalization of non-air pixel intensity values.

29. The method of claim 21, further comprising at least one of (i) randomly flipping the at least one image, (ii) randomly rotating the at least one image, (iii) randomly cropping the at least one image, or (iv) applying a random affine shear to the at least one image.

30. (canceled)

31. The method of claim 21, wherein the at least one neural network is a convolutional neural network (CNN).

32. The method of claim 31, wherein the CNN includes a plurality of layers.

33. The method of claim 32, wherein the CNN includes 15 hidden layers.

34. The method of claim 32, wherein the CNN includes five residual layers.

35. The method of claim 32, wherein the CNN includes at least one inception style layer after a ninth hidden layer.

36. The method of claim 32, wherein the CNN includes at least one fully connected layer after a 13^{th} layer thereof.

37. The method of claim 36, wherein the at least one fully connected layer includes 16 neurons.

38. The method of claim 32, wherein the CNN includes at least one linear layer after a 13^{th} layer.

39. The method of claim 38, wherein the at least one linear layer includes 8 neurons.

40. The method of claim 21, further comprising determining what action to perform or whether to perform any action based on the determined DCIS information.

41. A system for determining ductal carcinoma in situ (DCIS) information regarding at least one patient, comprising:
a computer hardware arrangement configured to:
receive at least one image of at least one internal portion of a breast of the at least one patient; and
automatically determine the DCIS information by applying at least one neural network to the at least one image.

42-60. (canceled)
